(19) United States
(12) Patent Application Publication
(10) Pub. No.: US 2025/0312561 A1
(43) Pub. Date: Oct. 9, 2025

(54) WAKING DEVICE USING VIBRATION OR SOUND AND METHOD THEREOF

(71) Applicant: BRLAB INC., Seoul (KR)

(72) Inventors: Jongmin LEE, Seoul (KR); Hyunbin KWON, Seoul (KR); Kwangsuk PARK, Seoul (KR); Byunghun CHOI, Seoul (KR); Jonghyeok JEONG, Seoul (KR)

(21) Appl. No.: 19/245,044

(22) Filed: Jun. 20, 2025

Related U.S. Application Data
(63) Continuation of application No. PCT/KR2023/021426, filed on Dec. 22, 2023.

(30) Foreign Application Priority Data
Dec. 22, 2022 (KR) 10-2022-0182003
Dec. 22, 2022 (KR) 10-2022-0182004
Apr. 10, 2023 (KR) 10-2023-0047031
Nov. 3, 2023 (KR) 10-2023-0151052

(51) Int. Cl. A61M 21/02; A61M 21/00

(52) U.S. Cl. CPC A61M 21/02; A61M 2021/0022; A61M 2021/0027; A61M 2021/0083

(57) ABSTRACT

The present disclosure relates to a waking device using vibration or sound and a method thereof, and more particularly, a waking device using vibration or sound according to an embodiment of the present disclosure may include at least one layer including a plurality of components; a detection unit provided in the layer unit to detect user state information; a memory unit in which user state information detected by the detection unit is stored; a control unit that generates a user-specific waking signal according to user state information stored in the memory unit; and a waking unit that generates user-specific stimulation according to a signal generated from the control unit.

### CROSS-REFERENCE TO RELATED APPLICATIONS

This application is a Continuation of Application No. PCT/KR2023/021426, filed on Dec. 22, 2023, which in turn claims the benefit of Korean Patent Applications No. 10-2022-0182003, filed on Dec. 22, 2022, No. 10-2022-0182004, filed on Dec. 22, 2022, No. 10-2023-0047031, filed on Apr. 10, 2023, and No. 10-2023-0151052, filed on Nov. 3, 2023. The entire disclosures of all these applications are hereby incorporated by reference.

### TECHNICAL FIELD

The present disclosure relates to a waking device using vibration or sound and a method thereof, and more particularly, to a waking device using vibration or sound that detects a user's state information present on a mattress and induces wake-up by outputting vibration or sound in a manner appropriate for the user based on the detected user information, and a method thereof.

### BACKGROUND ART

Recently, as people's living standards and quality have improved, the demand for ‘good sleep’ has increased, and in particular, the industry that provides various sleep-inducing devices or services using the latest scientific technologies is growing significantly. Accordingly, many devices and services are being commercialized to improve sleep quality, such as sleep care services that provide guidance on sleep environment, habits, and posture through consulting with experts, and services that monitor a sleep state by detecting a user's breathing sounds, or the like when wearing a wearable device. In addition, just as much as ‘good sleep’, ‘good wake-up’ also plays an important role in determining a person's condition, and just waking up from sleep at the exact time he or she wants may greatly contribute to physical and mental health throughout the day, and this may help improve efficiency in aspects such as studying or working.

As the positive effects of ‘good sleep’ and ‘good wake-up’ are highlighted as described above, busy modern people have come to expect good quality sleep and wake-up effects just by sleeping on a bed or mattress without any separate equipment, and in line with this trend, development of beds that include a function of inducing wake-up for the user in addition to a function of the bed or mattress itself is actively carried out.

However, a wake-up function of a bed according to the prior art is simply connected to a user terminal in a wired/wireless manner and provides wake-up through a sound alarm at a time set in the user terminal, and in this case, there is a disadvantage in that the user cannot wake up at the set time when the user terminal is turned off or the user is in a deep sleep and cannot hear the sound.

The present disclosure has been proposed to complement the disadvantage of the prior art, and provides waking device using vibration or sound that induces wake-up in a manner suitable for each user through vibration or sound output from a bed or mattress, instead of simply inducing wake-up through an alarm function of the user terminal, and a method thereof.

### DISCLOSURE OF INVENTION

### Technical Problem

An aspect of the present disclosure is to provide a waking device using vibration or sound that induces a user to wake up through a vibration or sound generation module built into a structure such as a mattress on which the user sleeps, and a method thereof.

An aspect of the present disclosure is to provide alarm stimulation to wake up a user in a sleep state, and to propose how to generate the stimulation, or a process to be performed prior to applying the stimulation, with the aim of creating a high-quality wake-up environment for the user.

In the process, an aspect of the present disclosure is to provide a waking device using vibration or sound that detects user state information, and wakes up the user in a manner suitable for each user according to the detected state information, and a method thereof.

Moreover, an aspect of the present disclosure is to provide a waking device using vibration or sound that detects state information for each user even when multiple users sleep, and allows to wake up individually in a manner suitable for each user according to detected state information, and a method thereof.

Meanwhile, technical problems of the present disclosure are not limited to the above-mentioned problems, and other technical problems which are not mentioned herein will be clearly understood by those skilled in the art from the description below.

### Technical Solution

A waking device using vibration or sound according to the present disclosure may include at least one layer including a plurality of components; a detection unit provided in the layer unit to detect user state information; a memory unit in which user state information detected by the detection unit is stored; a control unit that generates a user-specific waking signal according to user state information stored in the memory unit; and a waking unit that generates user-specific stimulation according to a signal generated from the control unit.

In addition, in the waking device using vibration or sound according to the present disclosure, the detection unit may include at least one sensor among a pressure sensor, a vibration sensor, a position sensor, an acoustic sensor, an infrared sensor, a motion sensor, and a facial recognition sensor.

In addition, in the waking device using vibration or sound according to the present disclosure, the memory unit may map and store user state information detected by the detection unit for each user.

In addition, in the waking device using vibration or sound according to the present disclosure, the memory unit may map and store a user-specific wake-up time.

In addition, in the waking device using vibration or sound according to the present disclosure, the control unit may adjust at least one of an interval, an intensity, a duration, and an intensity by time zone of a user-specific waking signal according to user state information.

In addition, in the waking device using vibration or sound according to the present disclosure, the control unit may adjust the on/off of a waking signal according to user feedback.

In addition, in the waking device using vibration or sound according to the present disclosure, the waking unit may include at least one of a vibration generation module and a sound generation module.

A waking method using vibration or sound according to the present disclosure, which is a waking method performed in a waking device including a layer unit, a detection unit, a memory unit, a control unit, and a waking unit, may include detecting user state information from a detection unit provided in the layer unit; storing user state information detected by the detection unit in a memory unit; generating a user-specific waking signal according to user state information stored in the memory unit from the control unit; and generating user-specific stimulation from the waking unit according to a signal generated from the control unit.

In addition, in the waking method using vibration or sound according to the present disclosure, the detecting of user state information may be performed to detect at least one information item among the user's weight, height, body proportions, identification information, and sleep state through a detection unit including at least one sensor among a pressure sensor, a vibration sensor, a position sensor, an acoustic sensor, an infrared sensor, a motion sensor, and a facial recognition sensor.

In addition, in the waking method using vibration or sound according to the present disclosure, the storing of user state information in the memory unit may be performed to map and store user state information detected by the detection unit for each user.

In addition, in the waking method using vibration or sound according to the present disclosure, the storing of user state information in the memory unit may be performed to map and store a user-specific wake-up time.

In addition, in the waking method using vibration or sound according to the present disclosure, the generating of a user-specific waking signal in the control unit may be performed to adjust at least one of an interval, an intensity, duration, and an intensity by time zone of a user-specific waking signal according to user state information.

In addition, in the waking method using vibration or sound according to the present disclosure, the generating of a user-specific waking signal in the control unit may be performed to adjust the on/off of the waking signal according to user feedback.

In addition, in the waking method using vibration or sound according to the present disclosure, the waking unit may include at least one of a vibration generation module and a sound generation module, and the generating of user-specific stimulation in the waking unit may be performed to output vibration or sound according to a user-specific waking signal generated by the control unit.

A waking device using vibration or sound according to another embodiment of the present disclosure may include at least one layer including a plurality of components; first and second detection units provided in the layer unit to detect a plurality of user state information; a memory unit in which a plurality of user state information detected by the first and second detection units are stored; a control unit that generates a user-specific waking signal according to a plurality of user state information stored in the memory unit; and first and second waking units that generate user-specific stimulation according to a signal generated from the control unit, wherein the first and second waking units are independently driven by the control unit so as to generate stimulation for each of a plurality of users.

In addition, in the waking device using vibration or sound according to another embodiment of the present disclosure, the memory unit may map and store a plurality of user state information detected by the first and second detection units for each user.

In addition, in a waking device using vibration or sound according to another embodiment of the present disclosure, the first and second waking units may include at least one of a vibration generation module or a sound generation module.

In addition, in the waking device using vibration or sound according to another embodiment of the present disclosure, the first and second waking units are generate stimulation with different intervals, intensities, durations, and time zones for the plurality of users, respectively.

### Advantageous Effects

According to the present disclosure, a user's wake-up may be induced through vibration or sound or vibration and sound stimulation, thereby effectively inducing the user's wake-up at a wake-up time targeted by the user.

In addition, according to the present disclosure, user state information may be detected, and an optimal waking signal may be generated for each detected user state information, thereby inducing the user's wake-up in the most effective manner for each user.

Moreover, according to the present disclosure, user feedback information on detected user state information or waking signals may be accumulated and recorded periodically/aperiodically and updated, and an optimal waking signal for each user may be generated according to the updated information, thereby inducing the user's wake-up in an optimal manner according to a change in the user's status even if the user does not specifically set his/her state information or preferred or disliked method.

Furthermore, the present disclosure may distinguish a plurality of users and detect user-specific state information, thereby generating a user-specific waking signal for the plurality of users through a single device.

Meanwhile, the effects of the present disclosure may not be limited to the above-mentioned effects, and other technical effects which are not mentioned herein will be clearly understood by those skilled in the art from the description below.

### BRIEF DESCRIPTION OF THE DRAWINGS

FIG. 1 is a diagram for explaining a waking device using vibration or sound according to the present disclosure.

FIG. 2 is a diagram for explaining a waking method using vibration or sound according to an embodiment of the present disclosure.

FIG. 3 is a diagram for explaining a waking method using vibration or sound according to another embodiment of the present disclosure.

FIG. 4 is a diagram for explaining a method of generating a waking signal based on a user's heart rate according to an embodiment of the present disclosure.

FIGS. 5A to 5E are diagrams for explaining a method of inducing wake-up by user-specific stimulation generated from a waking unit according to an embodiment of the present disclosure.

FIGS. 6A to 6C are diagrams for explaining a method of inducing wake-up by user-specific stimulation generated from a waking unit according to another embodiment of the present disclosure.

FIG. 7 is a diagram for explaining a user-specific waking device according to still another embodiment of the present disclosure.

### BEST MODE FOR CARRYING OUT THE INVENTION

The details of the objects and technical configurations of the present disclosure and operational effects thereof will be more clearly understood from the following detailed description based on the accompanying drawings appended hereto. Hereinafter, embodiments according to the present disclosure will be described in detail with reference to the accompanying drawings.

Embodiments disclosed herein should not be interpreted as limiting or used to limit the scope of the present disclosure. It is apparent for those skilled in the art that a description including embodiments herein has various applications. Therefore, any embodiments described in the detailed description of the present disclosure are illustrative for better understanding of the present disclosure and are not intended to limit the scope of the present disclosure to the embodiments.

Functional blocks illustrated in the drawings and described hereunder are only examples of possible implementations. In other implementations, other functional blocks may be used without departing from the concept and scope of the detailed description. Furthermore, one or more functional blocks of the present disclosure are illustrated as separate blocks, but one or more of the functional blocks of the present disclosure may be a combination of various hardware and software elements that execute the same function.

In addition, an expression that some elements are “included” is an expression of an “open type”, and the expression simply denotes that the corresponding elements are present, but should not be construed as excluding additional elements. Moreover, in case where it is mentioned that one element is “connected” or “coupled” to the other element, it should be understood that one element may be directly connected to the other element, but another element may be present therebetween.

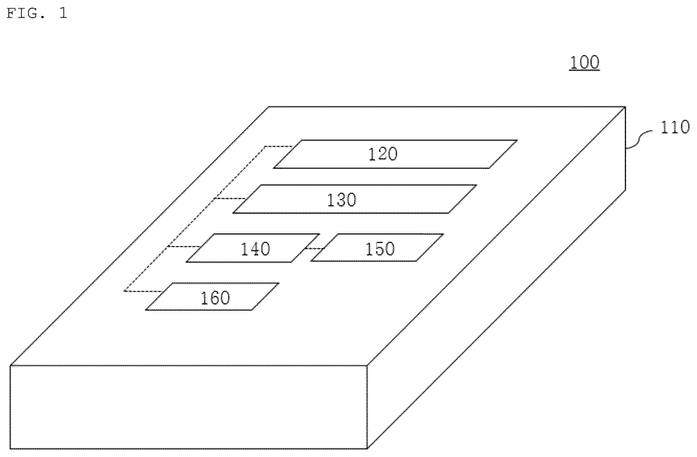

FIG. 1 is a diagram for explaining a waking device using vibration or sound according to the present disclosure.

Referring to FIG. 1, a waking device 100 according to the present disclosure includes at least one layer unit 110 having a plurality of components, a detection unit 120 provided in the layer unit 110 to detect user state information, a memory unit 130 in which the user state information detected by the detection unit 120 is stored, a control unit 140 that generates a user-specific waking signal according to the user state information stored in the memory unit 130, and a waking unit 150 that generates user-specific stimulation according to the signal generated from the control unit 140.

The layer unit 110 may be understood as a member having a receiving space in which the components to be described later can be arranged, and if a receiving space is provided, there is no limitation on the material or shape of the layer unit 110. The layer unit 110 may be a space where a user sleeps, for example, a mattress, or may be any one of a plurality of surfaces constituting a mattress. Additionally, the layer unit 110 may be a mat that may be placed on a mattress, and may furthermore be a member made of wood or metal rather than cotton with a fiber material. In this manner, if the layer unit 110 has a predetermined receiving space, there is no limitation on the material or shape. However, in order to help understand the disclosure, in this detailed description, the explanation will be continued assuming that the layer unit 110 is a mattress.

The detection unit 120, which is provided on the layer unit 110, is configured to detect and acquire state information from a user. The detection unit 120 may include various types of sensors that can detect user state information, such as a pressure sensor, a vibration sensor, a position sensor, an acoustic sensor, an infrared sensor, a motion sensor, a facial recognition sensor, and the like, and may be configured with a combination of at least one or more of the sensors.

Specifically, the detection unit 120 may detect the user's weight or position information through a pressure sensor, or detect the user's vibration signal through a vibration sensor to acquire state information such as a heart rate, a respiratory state, and a movement state. In addition, the detection unit 120 may recognize the user's voice through an acoustic sensor, and may detect the user's gesture through a motion sensor to receive feedback information.

In this way, the detection unit 120 of the present disclosure may be a component for detecting various state information of a user located on the layer unit 110, and adjusting the type, number, layout, and the like of sensors used to detect more precise and specific user state information cannot be limited to an embodiment of the present disclosure.

The memory unit 130, which is a component in which user state information detected by the detection unit 120 is stored, may be provided by being built into the waking device 100 or may be an external device connected to the waking device 100 through a wired or wireless communication manner.

In the memory unit 130, detected user state information may be mapped and stored for each user. For example, in addition to physical information such as a height and weight of user A, facial information, voice information, heart rate information, and the like, may be mapped and stored in the memory unit 130, and furthermore, specific information items such as heart rate information in a wake-up state, heart rate information in a sleep state, and a time taken from a sleep state to a wake-up state of user A may be mapped and stored. In addition, the memory unit 130 may store and map a user-specific wake-up time, thereby allowing the wake-up time to be set automatically or manually based on user information located in the layer unit 110.

The control unit 140 is a component that generates a wake-up signal that induces the user to wake up according to the user state information stored in the memory unit 130. For reference, the control unit 140 may also be understood as a central processing unit. The central processing unit may also be referred to as a controller, a microcontroller, a microprocessor, a microcomputer, or the like. Furthermore, the central processing unit may be implemented by hardware or firmware, software, or a combination thereof, and configured to include an application specific integrated circuit (ASIC) or a digital signal processor (DSP), a digital signal processing device (DSPD), a programmable logic device (PLD), or a field programmable gate array (FPGA) when implemented using hardware, and configured with firmware or software to include a module, a procedure, a function or the like that performs the foregoing functions or operations when implemented using firmware or software.

Here, the control unit 140 according to an embodiment of the present disclosure may generate a waking signal so as to induce wake-up according to a preset wake-up target time of a user, and in particular, adjust at least one of an interval, an intensity, a duration, and an intensity by time zone of a user-specific waking signal. That is, the present disclosure may not induce wake-up in the same way for all users, but rather may induce wake-up by a waking signal individually generated for each user by the control unit 140, thereby providing a more accurate and appropriate “good wake-up” effect to the user.

The waking unit 150, which is a component that generates user-specific stimulation according to a waking signal generated from the control unit 140, may include a vibration generation module or a sound generation module. The waking unit 150 may i) generate stimulation to induce the user's heart rate to reach a preset target value, or ii) generate an alarm stimulation to directly wake the user.

Meanwhile, the vibration generation module or sound generation module constituting the waking unit 150 may be driven to generate vibration stimulation or sound stimulation using a principle similar to that of a woofer speaker, and may be driven to output vibration and stimulation sound stimulation simultaneously when a stronger stimulation is desired. That is, the waking unit 150 of the present disclosure may induce the user to wake up in a manner suitable for the user or preferred by the user among vibration stimulation, sound stimulation, or vibration and sound stimulation. Here, the waking unit 150 according to an embodiment of the present disclosure, which is a component for providing a user with vibration or sound stimulation, or stimulation of a combination thereof, may only illustrate a woofer speaker as a device for generating the vibration or sound stimulation, and may be applied to any device that can generate vibration or sound or vibration and sound stimulation.

In addition, the waking device 100 of the present disclosure may further include a feedback receiving unit 160 that receives feedback from a user. The feedback receiving unit 160 may be implemented in various forms such as a contact or non-contact panel, an acoustic receiver, a motion recognizer, a button, and the like, and the user may transmit various information on the waking device 100 through the feedback receiving unit 160. For example, the user may adjust his or her desired wake-up time through the feedback receiving unit 160, and may also adjust the increase/decrease or on/off of the stimulation generated from the waking unit 150.

As described above, the waking device 100 according to the present disclosure may individually generate an optimal waking signal for each user according to the user state information by detecting the user state information through the detection unit 120 provided with various sensors. In addition, the waking device 100 according to the present disclosure may induce the user to wake up through vibration and/or sound stimulation according to a waking signal individually generated by the user, and may induce, by more finely adjusting an appropriate interval, intensity, and duration of stimulation for each user, the user to wake up in an optimal manner at his or her desired time, thereby providing a more accurate and pleasant “good wake-up” effect.

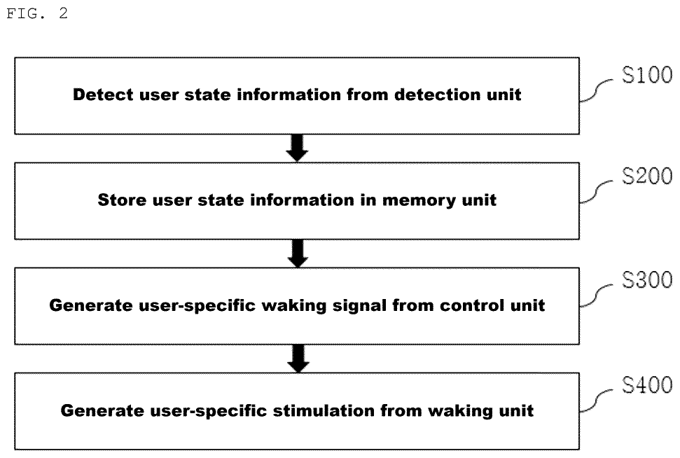

FIG. 2 is a diagram for explaining a waking method using vibration or sound according to an embodiment of the present disclosure.

Referring to FIG. 2, the present disclosure relates to a waking method performed in a waking device including a layer unit, a detection unit, a memory unit, a control unit, and a waking unit, which includes detecting user state information from a detection unit provided in the layer unit (S100), storing user state information detected by the detection unit in a memory unit (S200), generating a user-specific waking signal according to user state information stored in the memory unit from the control unit (S300), and generating user-specific stimulation from the waking unit according to a signal generated from the control unit (S400).

The main technical feature of the present disclosure is that stimulation for waking is generated according to state information of a user who sleeps on a layer unit, and first, the detecting of user state information in the detection unit (S100) is performed to detect at least one information item among the user's weight, body height, proportions, identification information, and sleep state through the detection unit including at least one sensor among a pressure sensor, a vibration sensor, a position sensor, an acoustic sensor, an infrared sensor, a motion sensor, and a facial recognition sensor.

The detection unit may detect not only the user's physical information, but also the user-specific identification level, such as face or voice, and may also detect how biometric information, such as a heart rate, changes depending on the user's wake-up or sleep state. Additionally, the detection unit may also detect where and in what posture the user sleeps. That is, the detecting of the user's state information (S100) is performed to detect various information of the user through various sensors provided in the detection unit.

Next, the storing of user state information detected by the detection unit (S200) in a memory unit is performed. The storing in the memory unit (S200) may be performed so as to allow the user state information detected by the detection unit to be mapped and stored for each user. For example, in addition to physical information such as a height and weight of user A, facial information, voice information, heart rate information, and the like, may be mapped and stored in the memory unit, and furthermore, specific information items such as heart rate information in a wake-up state, heart rate information in a sleep state, and a time taken from a sleep state to a wake-up state of user A may be mapped and stored. In addition, the memory unit may store and map a user-specific wake-up time, thereby allowing the wake-up time to be set automatically or manually based on user information located in the layer unit.

Next, the generating of a user-specific waking signal from the control unit according to user state information stored in the memory unit (S300) is performed. Specifically, the control unit may generate a waking signal so as to induce wake-up according to a preset wake-up target time of a user, and in particular, adjust at least one of an interval, an intensity, a duration, and an intensity by time zone of a user-specific waking signal.

That is, the waking method according to the present disclosure may not induce wake-up in the same way for all users, but rather may induce wake-up by a waking signal individually generated for each user by the control unit, thereby providing a more accurate and appropriate “good wake-up” effect to the user.

Next, the generating of user-specific stimulation from the waking unit according to a signal generated from the control unit (S400) is performed. The waking unit may include at least one of a vibration generation module and a sound generation module, and the generating of user-specific stimulation from the waking unit (S400) may be performed to output vibration or sound according to a user-specific waking signal generated from the control unit.

Specifically, the vibration generation module or sound generation module may refer to a module that can generate vibration or low-frequency sound (solid-state sound) having a long wavelength property, and may be driven to generate vibration stimulation or sound stimulation by a principle similar to that of a woofer speaker, for example. Additionally, if i stronger stimulation is required, vibration and sound stimulation may be output simultaneously. That is, the waking unit of the present disclosure may induce the user to wake up in a manner suitable for the user or preferred by the user among [vibration stimulation], [sound stimulation], and [vibration and sound stimulation].

As described above, the waking device method according

to the present disclosure may induce wake-up for each user in an accurate and effective manner through vibration or sound or vibration and sound stimulation by detecting user-specific state information and generating an optimal waking signal for each detected user state information.

Additionally, the disclosure may receive a feedback signal from the user as well as the detected user state information, and reflect them to generate a more optimized user-specific waking signal.

FIG. 3 is a diagram for explaining a waking method using vibration or sound according to another embodiment of the present disclosure, wherein referring to FIG. 3, the method may further include receiving user feedback information regarding stimulation generated in a waking unit (S500).

A user may transmit various feedback information to a waking device through various types of equipment such as a contact or non-contact panel, an acoustic receiver, a motion recognizer, and a button. For example, the user may adjust his or her desired wake-up time, or adjust the increase/decrease or on/off of the stimulation generated from the waking unit.

The user feedback information received in this manner may be transmitted to the memory unit so as to be stored by being mapped to the user information, and in this way, the control unit may generate a user-specific waking signal reflecting the user feedback information. In addition, since the process of receiving user feedback information is repeated several times and the user information is updated and stored, the user state information may always be kept up-to-date, and therefore, the present disclosure may generate an optimal waking signal suitable for the user's latest state information, thereby always providing a “good sleep” effect.

In addition, the present disclosure may first activate a sympathetic nervous system by generating stimulation at a sub-threshold level for a specific user to induce the user's heart rate to accelerate, and induce the user to wake up by pre-activating an autonomic nervous system in this manner. For reference, in this detailed description, the terms “sub-threshold level” and “threshold level” are used separately, wherein a “sub-threshold level” refers to a stimulation level at which a user's biosignal (especially heart rate) can be adjusted while the user is not yet awake, and a “threshold level” refers to a stimulation level at which a user can wake up from a sleep state. As can be inferred from the meaning of the term “threshold,” a “sub-threshold level” is a stimulation level defined at a boundary between a stimulation level at which the user's biosignal cannot be adjusted and a stimulation level at which the biosignal can be adjusted, and a “threshold level” is also a stimulation level defined at a boundary between a stimulation level at which the user can maintain a sleep state and a stimulation level at which the user can wake up. To facilitate understanding of the disclosure, stimulation at sub-threshold and threshold levels mentioned in this detailed description will be explained on the assumption that they are levels that can adjust a user's biosignal or levels that can wakeup the user, respectively, but it should be recalled that respective levels are defined as described above.

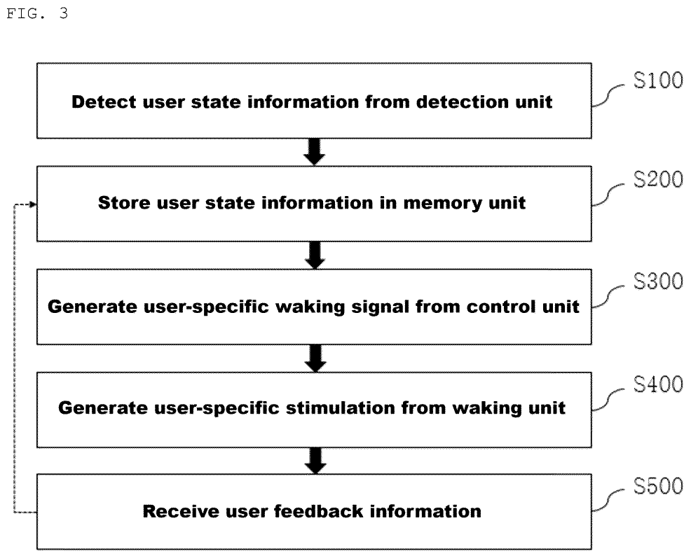

FIG. 4 is a diagram for explaining a method of generating a waking signal based on a user's heart rate according to an embodiment of the present disclosure, wherein the detection unit 120 in the waking device may detect the user's heart rate according to his or her state in order to derive user-specific stimulation at a sub-threshold level. The detection unit may detect the user's heart rate through a vibration sensor, and monitor or cumulatively record the heart rate according to the user's state, but an embodiment of the present disclosure is not limited thereto, and when heart rate information in more diverse states is required, the heart rate may be monitored through a separate terminal worn by the user, and the monitored heart rate information may be transmitted to the waking device of the present disclosure so as to be cumulatively recorded.

Specifically, the detection unit 120 in the waking device of the present disclosure may monitor the user's heart rate according to various states, and for example, may detect a heart rate in a normal non-sleep state, a heart rate while progressing from a non-sleep state to a sleep state, a heart rate in a sleep state, a heart rate while progressing from a sleep state to a wake-up state, a heart rate in a completely awake wake-up state, and the like, to accumulate and record them. The heart rate information may be monitored by detecting the heart rate at least once within a specific time interval and then calculating the average, and may also be monitored by excluding minimum and maximum values within the time interval and then calculating the average.

The waking device according to the present disclosure may define stimulation at a level where a heart rate changes according to the induction of randomly generated stimulation by a specific user as a sub-threshold level based on the heart rate information according to the user's state accumulated and recorded in this manner, and apply stimulation at the sub-threshold level so as to induce the user's heart rate in a sleep state to change to a set value. In addition, the waking device may define stimulation at a level that can wake up the user from sleep based on the user's accumulated and recorded state information as a threshold level, and change the user from a sleep state to a non-sleep state by applying stimulation at the threshold level. For reference, the stimulation at a sub-threshold level may be adjusted in intensity so as to allow an output of the stimulation to gradually increase in intensity until reaching the threshold level, and the output of the stimulation may be controlled in intensity so as to allow the signal to appear to change continuously. That is, the present disclosure may monitor the user's heart rate according to his or her normal sleep state or sleep stage, and in particular, determine the heart rate and stimulation level when a specific user wakes up from sleep, as well as the heart rate and stimulation level at that time to which the heart rate can be adjusted in a stage prior to waking up from sleep, thereby outputting vibration and/or sound stimulation so as to allow the user's heart rate (or other biosignals that can induce the user's wake-up behavior) to reach a predetermined value at a wake-up time set by the user or earlier than the set wake-up time.

For example, when a heart rate of a specific user upon waking up from sleep is between 80 and 85, and a heart rate of a current user during a sleep stage prior to waking up is monitored to be maintained between 70 and 75 for a predetermined period of time prior to waking up from sleep, a waking device according to the present disclosure may induce the user's heart rate to gradually increase by applying stimulation at a pre-defined sub-threshold level, and when the above specific user has set 6:30 as a wake-up time, the waking device may output vibration and/or sound stimulation so as to allow the user's heart rate to reach 80 by at least 6:20, which is 10 minutes earlier than 6:30. As to how much earlier than the set wake-up time to apply stimulation at a sub-threshold level to induce a set heart rate, the waking device may follow a calculation result based on its own calculation, but a calculation algorithm may be preferably implemented so as to induce a heart rate at a level at which the user can wake up by 0 to 5% earlier than the user's total sleep time, or so as to strengthen the stimulation to a threshold level, which is an intensity at a level at which the user can wake up. This is to give the user some time to maintain a sleep stage at a heart rate that is most suitable for the user during the sleep stage just prior to waking up, thereby preventing the user from experiencing an uncomfortable wake-up situation caused by instability of an autonomic nervous system at least at the moment of waking up.

For another example, in order to quickly induce a heart rate of a specific user currently being monitored, a waking device according to the present disclosure may apply vibration stimulation n % shorter or m % longer compared to the current heart rate, and in this manner, the waking device may apply stimulation at a sub-threshold level to the user using a current heart rate value as a reference value so as to induce the user's sleep state or autonomic nervous system to a desired state.

Meanwhile, such user heart rate information may be updated and accumulated periodically or aperiodically to derive the latest sub-threshold level or threshold level reflecting the user's surrounding environment or recent health condition may be derived, and therefore, the present disclosure may expect a “good wake-up” effect in a manner most suitable for the user without the user having to separately input his or her latest environment information or health condition.

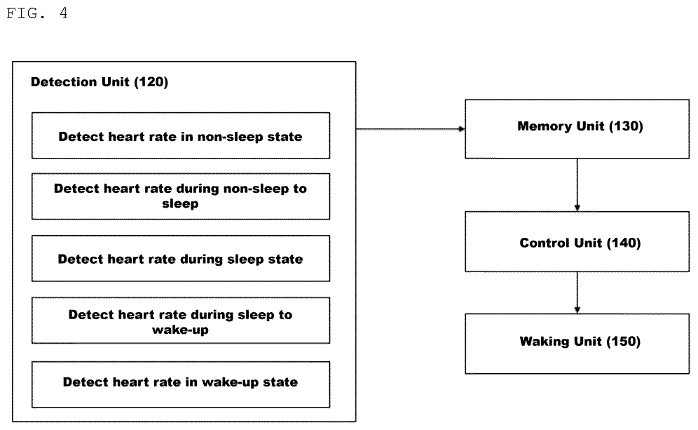

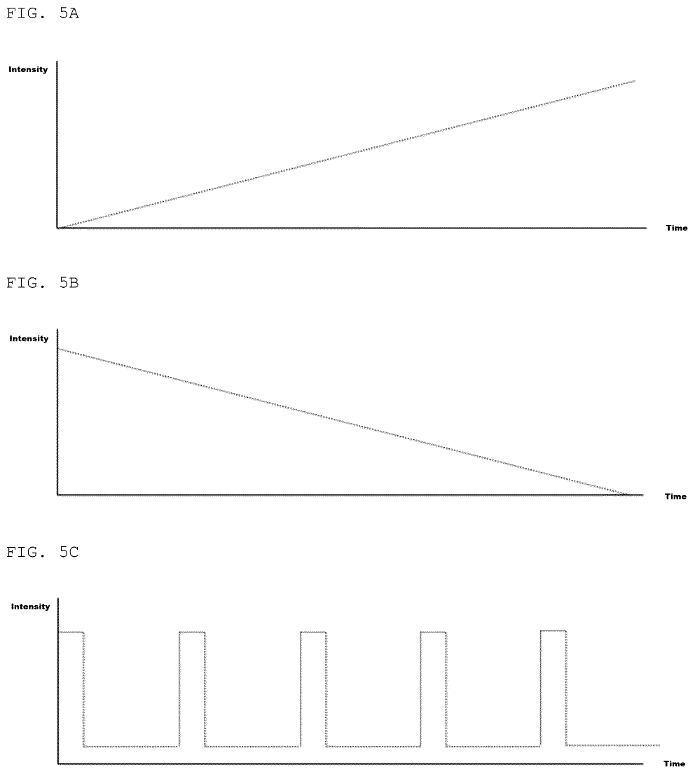

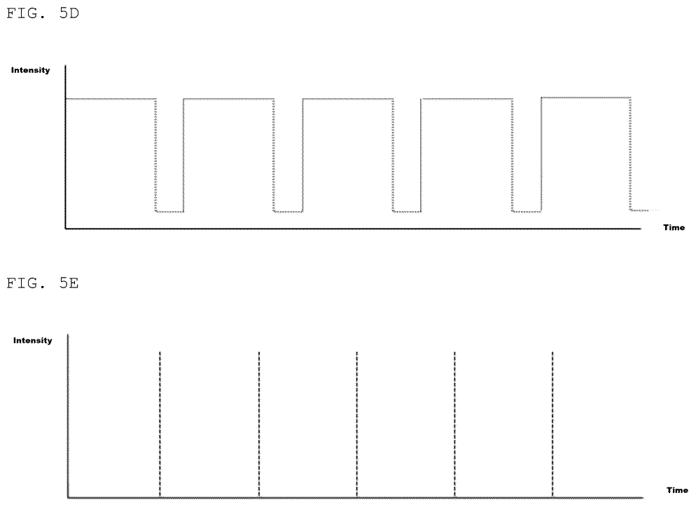

Moreover, the present disclosure may provide a waking signal and an intensity or time of stimulation optimized for each user, and FIGS. 5A to 5D are diagrams for explaining a method of inducing wake-up by a user-specific stimulation generated from a waking unit according to an embodiment of the present disclosure.

A magnitude and duration of stimulation that induces an appropriate wake-up state may vary depending on the user's tendency, preference, and sleep state, and the present disclosure may generate an optimal waking signal for each user from a control unit based on user state information acquired from a detection unit.

For example, an intensity provided to the user may be gradually or stepwise increased or decreased to induce wake-up, wherein as shown in FIG. 5A, the user may be awakened by starting with weak stimulation and then gradually generating stronger stimulation, or the user may be awakened by starting with strong stimulation and then gradually generating weaker stimulation, as shown in FIG. 5B.

In addition, the strength and weakness of stimulation provided to the user may be intermittently adjusted to induce wake-up in an interval manner, wherein the user's wake-up can be induced in an interval manner that generates strong stimulation for a short period of time followed by weak stimulation for a long period of time as shown in FIG. 5C, and the user's wake-up can be induced in an interval manner that generates strong stimulation for a long period time followed by weak stimulation for a short period of time as shown in FIG. 5D. Additionally, the user's wake-up may be induced by generating an impulse signal at set intervals or at set points in time.

Moreover, although not shown, the present disclosure may be implemented in various ways, such as a method of continuously generating only a strong intensity or continuously generating only a weak intensity, a method of simultaneously generating strong vibration stimulation and weak sound stimulation or simultaneously generating weak vibration stimulation and strong sound stimulation, a method of generating strong vibration and sound stimulation or weak vibration and sound stimulation, a method of generating only vibration stimulation and then generating sound stimulation at the end or beginning, a method of generating only sound stimulation and then generating vibration stimulation at the end or beginning, and the like.

In other words, the present disclosure may generate a most optimal waking signal for each user by generating a waking signal based on the user's state information, rather than generating vibration or sound stimulation in a uniform or fixed manner set by the user to induce the user to wake up. Furthermore, the present disclosure may provide a high-quality “good wake-up” state by periodically/aperiodically updating the user's state information, likes/dislikes, or the like, and generating a user-specific waking signal based on the updated information so as to generate an optimal waking signal at any time according to the user's condition and sleep state.

In addition, as another embodiment of the present disclosure, it may be also possible to induce wake-up by generating a user-specific waking signal at a wake-up target time set by the user or before or after the wake-up target time.

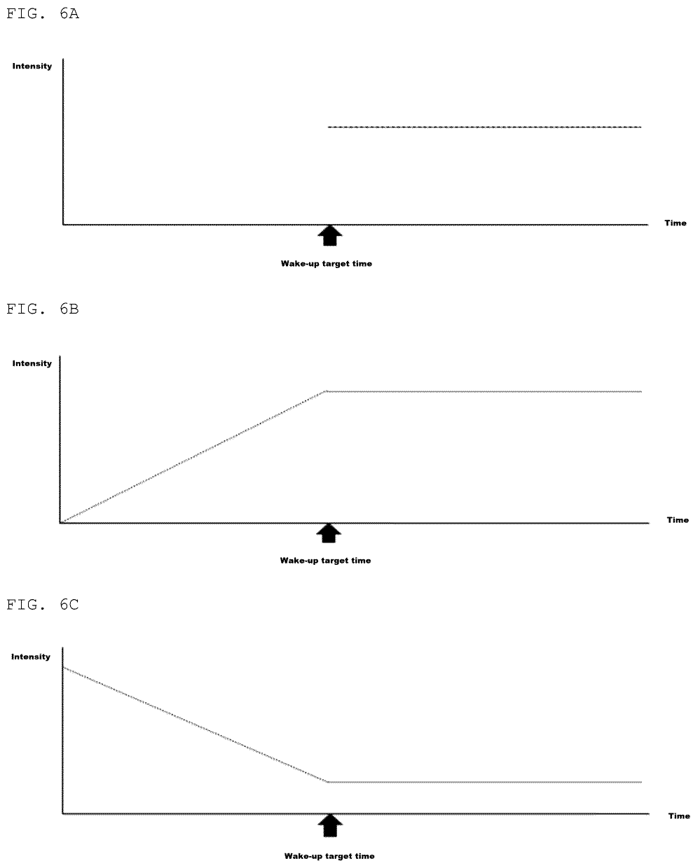

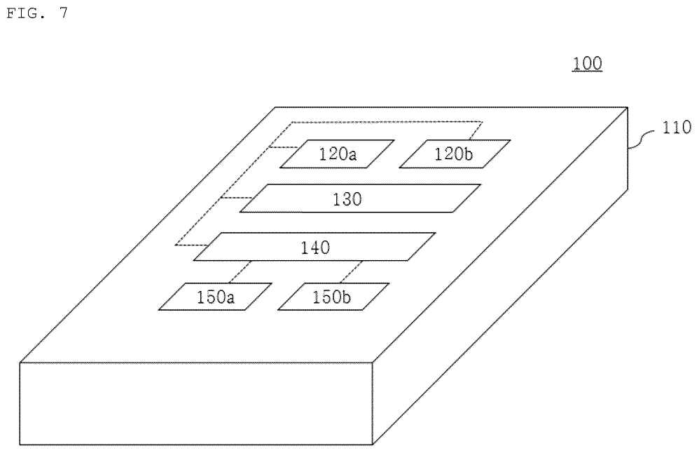

FIGS. 6A to 6C are diagrams for explaining a method of inducing wake-up by user-specific stimulation generated from a waking unit according to another embodiment of the present disclosure, wherein vibration or sound or vibration and sound stimulation may be generated from a wake-up target time set by a user to induce wake-up to the user as shown in FIG. 6A, wake-up may be induced in such a manner that stimulation is generated with a weak intensity at first and then strong stimulation is generated from a wake-up target time set by the user as shown in FIG. 6B, and wake-up may also be induced in such a manner that stimulation is generated with a strong intensity at first and then weak stimulation is generated from a wake-up target time set by the user as shown in FIG. 6C.

That is, the present disclosure may induce the user to wake up in various manners, such as in a manner that generates a waking signal from a wake-up target time set by the user, or in a manner that generates strong or weak stimulation before or after the wake-up target time, and therefore, the present disclosure may adjust a stimulation generation time and intensity in an optimal manner according to preference of each user and/or state information of each user.

In addition, although not shown, the present disclosure may be implemented in various embodiments, such as adjusting an intensity of stimulation stepwise or gradually at a wake-up target time set by the user or before or after the wake-up target time, or repeatedly providing vibration or sound or vibration and sound stimulation, and an optimal waking signal for each user may be changed and applied in real time according to the user's feedback information or user state information that is updated periodically/aperiodically.

Meanwhile, a waking device according to still another embodiment of the present disclosure may induce wake-up by generating an optimal waking signal for each user, even when used by a plurality of users.

FIG. 7 is a diagram for explaining a user-specific waking device according to still another embodiment of the present disclosure, wherein a component for detecting user-specific state information and a component that generates user-specific stimulation to induce wake-up may be respectively configured in plurality for each user. Hereinafter, a description of a component that performs the same or similar function as that of FIG. 1 will be omitted, and a description of a waking device according to still another embodiment of the present disclosure will be given.

As shown in FIG. 7, the waking device 100 using vibration or sound according to still another embodiment of the present disclosure includes at least one layer unit 110 having a plurality of components, first and second detection units 120a, 120b provided in the layer unit 110 to detect a plurality of user state information, a memory unit 130 in which a plurality of user state information detected by the first and second detection units 120a, 120b is stored, a control unit 140 that calculates to distinguish which user each of a plurality of users is based on the state information detected by the first and second detection units, and generates a user-specific waking signal according to the plurality of user state information stored in the memory unit 130, and first and second waking units 150a, 150b that generate stimulation for each user according to the signal generated from the control unit 140.

Here, the first and second detection units 120a, 120b, which are configured to detect a plurality of user-specific state information, may detect identification information that can distinguish users located in the layer unit 110, state information for each user, and a position of the user. A process of detecting identification information that can distinguish users may be carried out while the users are sleeping on the layer unit 110, and when considering that the position of users is unlikely to change in a sleep state, the process may preferably be performed once in the sleep state, but may also be performed repeatedly at least twice depending on the user's characteristics or requests.

There are various methods that can be applied to distinguish the characteristics and positions of a plurality of users, and for example, which may be performed by detecting the user's body information such as a height, a weight, and a shoulder width through a pressure sensor provided in the first and second detection units 120a, 120b, or by detecting a magnitude of a pressure, a weight, and an impact strength applied when the user comes up on the layer unit 110.

In addition, in order to distinguish the characteristics and positions of a plurality of users, it may be performed by detecting the user's voice, or the like, through an acoustic sensor provided in the waking device 100, or detecting the user's face, body part, or the like, through an image sensor provided in the waking device 100. Alternatively, a plurality of users may be distinguished by pre-stored user-specific voice signals, and for example, if user A is stored with a voice signal of “I'm going to sleep” and user B is stored with a voice signal of “I'm going to lie down,” user A and user B may be distinguished by detecting those voice signals.

In addition, in order to distinguish the characteristics and positions of a plurality of users, a user-specific sleep pattern or biometric information may be detected, and for example, the characteristics and positions of users may be distinguished by detecting various biometric information such as change in sleeping position and a frequency of changes, frequently taken sleeping positions, a heart rate pattern, and a breathing rate pattern.

Here, various sensors that can distinguish the characteristics and positions of a plurality of users may be provided as a component of the first and second detection units 120a, 120b, may be provided in a specific part of the waking device 100, or may be provided as a separate device connected to the waking device 100 in a wired/wireless manner. In addition, in order to distinguish the characteristics and positions of a plurality of users, at least one or more of the various methods mentioned above may be applied in combination, and methods that can most accurately distinguish depending on the accuracy of user classification may also sequentially applied.

The state information of a plurality of users detected by the first and second detection units 120a, 120b may be stored in the memory unit 130, and the memory unit 130 may map and store state information and positions for the plurality of users, respectively. Additionally, information items stored in the memory unit 130 may be updated periodically/aperiodically for higher accuracy.

The control unit 140 generates a user-specific waking signal based on a plurality of user state information stored in the memory unit 130. In particular, in the present disclosure, as a plurality of user-specific state information items are mapped and stored for each user in the memory unit 130, the control unit 140 may generate an optimal waking signal for each of the plurality of users.

In the first and second waking units 150a, 150b, user-specific stimulation is generated according to a user-specific waking signal generated from the control unit 140. The first and second waking units 150a, 150b may include at least one of a vibration generation module and a sound generation module, and may be driven to output vibration stimulation and sound stimulation simultaneously when stronger stimulation is desired. That is, the waking unit 150 of the present disclosure may induce the user to wake up in a manner suitable for the user or preferred by the user among vibration stimulation, sound stimulation, or vibration and sound stimulation.

Here, the waking units 150a, 150b according to still another embodiment of the present disclosure are configured to provide a user with vibration or sound stimulation, or a stimulation of a combination thereof, wherein any device that can generate vibration or sound stimulation, or vibration and sound stimulation may be applied, and different modules may also be configured for a plurality of users, respectively.

Moreover, the first and second waking units 150a, 150b according to still another embodiment of the present disclosure may be independently driven by the control unit 140, and therefore, the present disclosure may generate stimulation at different intensities and durations at different times for a plurality of users, respectively. That is, a stimulation signal that induces a “good wake-up” state may be different for each of a plurality of users, and in the case of the present disclosure, since the first and second waking units 150a, 150b are controlled to be independently driven, stimulation may be differently provided for each user. In other words, in still another embodiment of the present disclosure, various embodiments of the waking method shown and described above in FIGS. 1 to 6 are independently applied to a plurality of users, respectively.

As described above, the waking device 100 according to still another embodiment of the present disclosure may be provided with first and second detection units 120a, 120b that detect a plurality of user state information, thereby not only allowing for recognition and distinction of each user, but also allowing for sleep pattern analysis or authentication of each user. Moreover, in the waking device 100 according to still another embodiment of the present disclosure, first and second waking units 150a, 150b that generate waking stimulation for a plurality of users may be independently driven, and thus even if user-specific sleep patterns, wake-up target times, and preferred wake-up methods are different, optimal waking stimulation may generated for each user, thereby providing a “good wake-up” effect to all users in an optimal manner at their desired times.

For reference, some specific examples of inducing a user to wake up using a waking device according to the present disclosure will be described as follows.

In one embodiment, a user may set an alarm time of a waking device to 6:00, and the waking device may generate stimulation at least at 6:00 sharp that increases an intensity of the stimulation to a level that reliably wakes up the user, thereby allowing the user to wake up at 6:00. At this time, the intensity of the stimulation may be adjusted to a maximum limit that can be output by the vibration generation module or the sound generation module, and even if it is not the maximum limit, the stimulation intensity may be output at least 20% higher than the threshold level, which is the stimulation intensity that can wake up the user, thereby ensuring that the user wakes up. Additionally, the waking device may drive the vibration generation module and the sound generation module simultaneously to further increase the intensity of their combined output.

In another embodiment, the waking device may be designed to apply stimulation starting from a time “n” minutes before an alarm time set by the user, and the intensity of the stimulation may be gradually increased during the “n” minutes so as not to allow the user to be startled by a sudden alarm signal output at the set time. Additionally, the intensity of the stimulation at this time may be adjusted so as to allow the user's biosignal (such as a heart rate) to gradually increase based on information from monitoring the user's biosignal. When the intensity of stimulation is gradually increased in this manner, it is expected that the user can naturally transition from a sleep state to a non-sleep state, thereby providing a good wake-up environment.

In still another embodiment, the waking device may be controlled to start monitoring the user's heart rate from a time “a” minute before the set alarm time, and generate and output vibration stimulation that is m % faster than the monitored heart rate (e.g., if an average of the monitored heart rate is 60 bpm (heartbeat interval is 1 second), assuming that the waking device generates vibration stimulation that is 5% faster, pulsed vibration stimulation with an interval of 0.95 seconds is generated) to induce the user's heart rate to a constant value. In this case, the user's heart rate may gradually increase in accordance with the vibration stimulation, and as the heart rate gradually increases, the waking device may generate vibration stimulation that is n % faster in response to the increased value, thereby adjusting the user's heart rate in this manner. At this time, the above “n” value may preferably be a real number smaller than “m”, but is not necessarily limited thereto, and the “n” value may have the same value as “m”.

In the above, a waking device using vibration or sound according to the present disclosure and a method thereof have been described. Meanwhile, the present disclosure is not limited to the foregoing specific embodiments and application examples, it will be of course understood by those skilled in the art that various modifications may be made without departing from the gist of the present disclosure as defined in the following claims, and it is to be noted that those modifications should not be understood individually from the technical concept and prospect of the present disclosure.

In particular, configurations that implement the technical features of the present disclosure included in the block diagrams and flowcharts shown in the drawings attached to this specification represent logical boundaries between the configurations. However, according to an embodiment of software or hardware, the shown configurations and functions thereof are executed in the form of stand-alone software modules, monolithic software structures, codes, services, and combinations thereof, and the functions may be implemented by being stored in a medium executable on a computer provided with a processor capable of executing the stored program codes, instructions, and the like, and therefore, all of these embodiments should also be regarded as falling within the scope of the present disclosure.

Accordingly, the accompanying drawings and technologies thereof describe the technical characteristics of the present disclosure, but should not be simply inferred unless a specific array of software for implementing such technical characteristics is clearly described otherwise. That is, the aforementioned various embodiments may be present, and may be partially modified while having the same technical features as those of the present disclosure, and thus such modified embodiments should also be regarded as falling within the scope of the present disclosure.

Furthermore, the flowchart describes operations in the drawing in a specific sequence, but has been shown to obtain the most preferred result, and it should not be understood that such operations must be carried out in the specific sequence or sequential sequence shown, or that all shown operations must be carried out. In a specific case, multi-tasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

1. A waking device, the device comprising:
at least one layer including a plurality of components;
a detection unit provided in the layer unit to detect user state information;
a memory unit in which user state information detected by the detection unit is stored;
a control unit that generates a user-specific waking signal according to user state information stored in the memory unit; and
a waking unit that generates user-specific stimulation according to a signal generated from the control unit.

2. The waking device of claim 1, wherein the memory unit maps and stores user state information detected by the detection unit for each user.

3. The waking device of claim 1, wherein the control unit generates stimulation at a sub-threshold level or threshold level that can be derived from accumulated user state information as the waking signal,
wherein the sub-threshold level is a critical level of stimulation that can induce a bio signal of a user in a sleep state to change to a set value, and
wherein the threshold level is a critical level of stimulation that can induce a user to wake up from sleep.

4. The waking device of claim 1, wherein the waking unit comprises:
at least one of a vibration generation module and a sound generation module.

5. A waking method performed in a waking device comprising a layer unit, a detection unit, a memory unit, a control unit, and a waking unit, the method comprising:
detecting user state information from a detection unit provided in the layer unit;
storing user state information detected by the detection unit in a memory unit;
generating a user-specific waking signal according to user state information stored in the memory unit from the control unit; and
generating user-specific stimulation from the waking unit according to a signal generated from the control unit.

6. A waking device, the device comprising:
at least one layer including a plurality of components;
first and second detection units provided in the layer unit to detect a plurality of user state information;
a memory unit in which a plurality of user state information detected by the first and second detection units are stored;
a control unit that generates a user-specific waking signal according to a plurality of user state information stored in the memory unit; and
first and second waking units that generate user-specific stimulation according to a signal generated from the control unit,
wherein the first and second waking units are independently driven by the control unit so as to generate stimulation for each of a plurality of users.

7. The waking device of claim 6, wherein the memory unit maps and stores a plurality of user state information detected by the first and second detection units for each user.

8. The waking device of claim 6, wherein the control unit executes an operation to determine which user each of the plurality of users is based on the plurality of user state information acquired by the first and second detection units.

9. A waking method performed in a waking device comprising a layer unit, first and second detection units, a memory unit, a control unit, and first and second waking units, the method comprising:
detecting user state information items of a plurality of users from the first and second detection units provided in the layer unit;
storing user state information items detected by the detection units in the memory unit;
generating a waking signal for each user according to the user state information items stored in the memory unit from the control unit; and
generating user-specific stimulation from at least one of the first or second waking units according to a signal generated from the control unit.

10. The waking method of claim 9, wherein the generating of a waking signal for each user from the control unit is executed subsequent to a process of determining which user each of plurality of users is based on a plurality of user state information acquired by the first and second detection units.
